(19) United States
(12) Patent Application Publication
(10) Pub. No.: US 2023/0093375 A1
(43) Pub. Date: Mar. 23, 2023

(54) SURGICAL ROBOTIC SYSTEMS

(71) Applicant: Covidien LP, Mansfield, MA (US)

(72) Inventors: Kevin Desjardin, Prospect, CT (US); Astley C. Lobo, West Haven, CT (US)

(21) Appl. No.: 17/922,683

(22) PCT Filed: May 3, 2021
(86) PCT No.: PCT/US2021/030397

§ 371 Date: Nov. 1, 2022

Related U.S. Application Data
(60) Provisional application No. 63/021,721, filed on May 8, 2020.

(51) Int. Cl. A61B 34/30; F16H 9/24

(52) U.S. Cl. CPC A61B 34/30; F16H 9/24; A61B 2034/305

(57) ABSTRACT

A surgical robotic system includes a surgical robotic arm and a surgical instrument configured to couple to the surgical robotic arm. The surgical robotic arm has a housing and a pulley rotationally supported in a distal end portion of the housing. The surgical instrument has a housing and a gear rotationally supported in a proximal end portion of the housing thereof. The gear is configured to operably couple to the pulley of the surgical robotic arm to transfer rotational motion from the pulley of the surgical robotic arm to a functional component of the surgical instrument.

### FIELD

The present technology is generally related to surgical robotic systems used in minimally invasive medical procedures.

### BACKGROUND

Some surgical robotic systems included a console supporting a surgical robotic arm and a surgical instrument or at least one end effector (e.g., a surgical clip applier, stapler, or a grasping tool) mounted to the robotic arm. The robotic arm provided mechanical power to the surgical instrument for its operation and movement. The surgical instrument may have been configured to be detachably coupled to an end portion of the robotic arm. Upon coupling the surgical instrument to the end portion of the robotic arm, one or more mechanical driving components in the robotic arm may have been operably coupled to a respective mechanically driven component of the surgical instrument.

### SUMMARY

In one aspect of the present disclosure, a surgical robotic system for use in a minimally invasive surgical procedure is provided. The surgical robotic system includes a surgical robotic arm and a surgical instrument each having a housing. The surgical robotic arm has a first pulley rotationally supported in a proximal end portion of the housing, a second pulley rotationally supported in a distal end portion of the housing, and a belt operably coupling the first and second pulleys to one another. The housing of the surgical instrument has a proximal end portion configured to couple to the distal end portion of the housing of the surgical robotic arm. The surgical instrument includes a gear rotationally supported in a proximal end portion of the housing and configured to operably couple to the second pulley of the surgical robotic arm. The gear is configured to transfer rotational motion from the first pulley of the surgical robotic arm to a functional component of an end effector of the surgical instrument.

In aspects, the second pulley may be a compound gear including a first gear and a second gear non-rotatably fixed to the first gear. The first gear may be operably engaged to the belt and configured to rotate in response to movement of the belt. The second gear may be configured to operably engage the gear of the surgical instrument.

In aspects, the gear of the surgical instrument may protrude proximally from the proximal end portion of the housing of the surgical instrument and/or the second gear of the surgical robotic arm may protrude distally from the distal end portion of the housing of the surgical robotic arm.

In aspects, the surgical instrument may further include a linear actuator supported in the housing of the surgical instrument and configured to move axially within the housing in response to a rotation of the gear.

In aspects, the linear actuator may have a proximal end portion operably engaged with the gear, and a distal end portion configured to couple to the functional component of the end effector.

In aspects, the proximal end portion of the linear actuator may have a plurality of gear teeth in meshing engagement with the gear.

In aspects, the surgical robotic arm may further include a motor drivingly coupled to the first pulley and configured to rotate the first pulley.

In aspects, the distal end portion of the housing of the surgical robotic arm may have a male or female mechanical mating feature. The proximal end portion of the housing of the surgical instrument may have the other of the male or female mechanical mating feature configured to matingly engage the male or female mechanical mating feature of the distal end portion of the housing of the surgical robotic arm.

In accordance with another aspect of the disclosure, a surgical robotic system for use in a minimally invasive surgical procedure is provided. The surgical robotic system includes a surgical robotic arm and a surgical instrument each including an elongated housing. The surgical robotic arm includes a pulley and a compound gear. The pulley is rotationally supported in a proximal end portion of the housing. The compound gear is rotationally supported in the distal end portion of the housing and operably engaged to the pulley. The housing of the surgical instrument housing has a proximal end portion configured to detachably couple to the distal end portion of the housing of the surgical robotic arm. The surgical instrument includes an annular gear and a linear actuator. The annular gear is rotationally supported in the proximal end portion of the housing of the surgical instrument and configured to operably couple to the compound gear of the surgical robotic arm. The linear actuator is supported in the housing of the surgical instrument and has a proximal end portion and a distal end portion. The proximal end portion of the linear actuator is operably engaged to the annular gear, and the distal end portion of the linear actuator is configured to couple to a functional component of an end effector of the surgical instrument. The linear actuator is configured to move axially within the housing of the surgical instrument in response to a rotation of the pulley of the surgical robotic arm when the surgical instrument is coupled to the surgical robotic arm.

In aspects, the compound gear may include a first gear operably coupled to the pulley, and a second gear non-rotatably fixed to the first gear and configured to operably engage the annular gear of the surgical instrument.

In aspects, the surgical robotic arm may further include a belt wrapped around and engaged to the pulley and the first gear. The belt may be configured to transfer rotational motion of the pulley to the compound gear.

In aspects, the annular gear may protrude proximally from the proximal end portion of the housing of the surgical instrument and/or the second gear may protrude distally from the distal end portion of the housing of the surgical robotic arm.

In aspects, the proximal end portion of the linear actuator may have a plurality of gear teeth in meshing engagement with the annular gear.

In aspects, the surgical robotic arm may further include a motor drivingly coupled to the pulley and configured to rotate the pulley.

Further details and aspects of exemplary embodiments of the present disclosure are described in more detail below with reference to the appended figures.

As used herein, the terms parallel and perpendicular are understood to include relative configurations that are substantially parallel and substantially perpendicular up to about + or −10 degrees from true parallel and true perpendicular.

### BRIEF DESCRIPTION OF THE DRAWINGS

Embodiments of the present disclosure are described herein with reference to the accompanying drawings, wherein:

FIG. 1 is a schematic illustration of a surgical robotic system including a surgical robotic arm coupled with a surgical instrument in accordance with the present disclosure;

FIG. 2A is a perspective view of the surgical robotic arm and surgical instrument of FIG. 1 in a decoupled state;

FIG. 2B is a front view of the surgical robotic arm and surgical instrument of FIG. 2A in a coupled state;

FIG. 3 is a perspective view illustrating internal components of the surgical robotic arm and surgical instrument of FIG. 2A;

FIG. 4 is a front view illustrating the internal components of the surgical robotic arm and surgical instrument of FIG. 2B; and

FIG. 5 is a perspective view, with parts separated, of an end effector of the surgical instrument.

### DETAILED DESCRIPTION

Embodiments of the presently disclosed surgical robotic system are described in detail with reference to the drawings, in which like reference numerals designate identical or corresponding elements in each of the several views. As used herein the term “distal” refers to that portion of the surgical robotic system or component thereof, that is closer to a patient, while the term “proximal” refers to that portion of the surgical robotic system or component thereof, that is further from the patient.

As will be described in detail below, provided is a surgical robotic system including a surgical robotic arm and a surgical instrument (e.g., a clip applier) that detachably couples to the surgical robotic arm. The surgical robotic arm has a pulley system that is actuated by a motor in the robotic arm, and the surgical instrument has a gear configured to operably couple to the pulley system of the robotic arm upon coupling the surgical instrument to the robotic arm. The surgical instrument also has a linear actuator operably engaged to the gear and coupled to a functional component (e.g., a spindle) of an end effector of the surgical instrument. The design of the robotic arm and surgical instrument enables an easy operable connection of the surgical instrument and its driven components with the robotic arm and its driving components. The present disclosure provides other advantages that may be apparent to one of ordinary skill in the art.

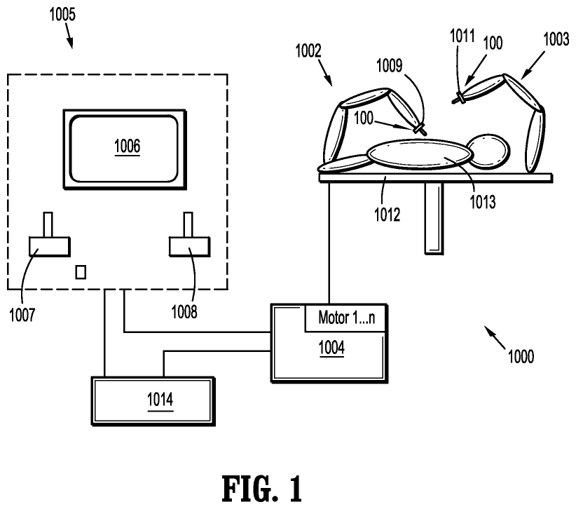

Referring to FIG. 1, a medical work station is shown generally as a surgical robotic system or work station 1000 and generally may include a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person (not shown), for example a surgeon, may be able to telemanipulate robot arms 1002, 1003 in a first operating mode.

Each of the robot arms 1002, 1003 may include a plurality of members, which are connected through joints, and an attaching device 1009, 1011, to which may be attached, for example, a surgical instrument 100 (e.g., a surgical clip applier), in accordance with any one of several embodiments disclosed herein, as will be described in greater detail below.

Robot arms 1002, 1003 may be driven by electric drives (not shown) that are connected to control device 1004. Control device 1004 (e.g., a computer) may be set up to activate the drives, in particular by means of a computer program, in such a way that robot arms 1002, 1003, their attaching devices 1009, 1011 and thus the surgical instrument 100, execute a desired movement according to a movement defined by means of manual input devices 1007, 1008. Control device 1004 may also be set up in such a way that it regulates the movement of robot arms 1002, 1003 and/or of the drives.

Medical work station 1000 may be configured for use on a patient 1013 lying on a patient table 1012 to be treated in a minimally invasive manner by means of the surgical instrument 100. Medical work station 1000 may also include more than two robot arms 1002, 1003, the additional robot arms likewise being connected to control device 1004 and being telemanipulatable by means of operating console 1005. A surgical instrument, such as, for example, a clip applier 100, may also be attached to the additional robot arm. Medical work station 1000 may include a database 1014, in particular coupled to with control device 1004, in which are stored, for example, pre-operative data from patient/living being 1013 and/or anatomical atlases.

Reference is made herein to U.S. Pat. No. 8,828,023, the entire content of which is incorporated herein by reference, for a more detailed description of the construction and operation of an exemplary surgical robotic system.

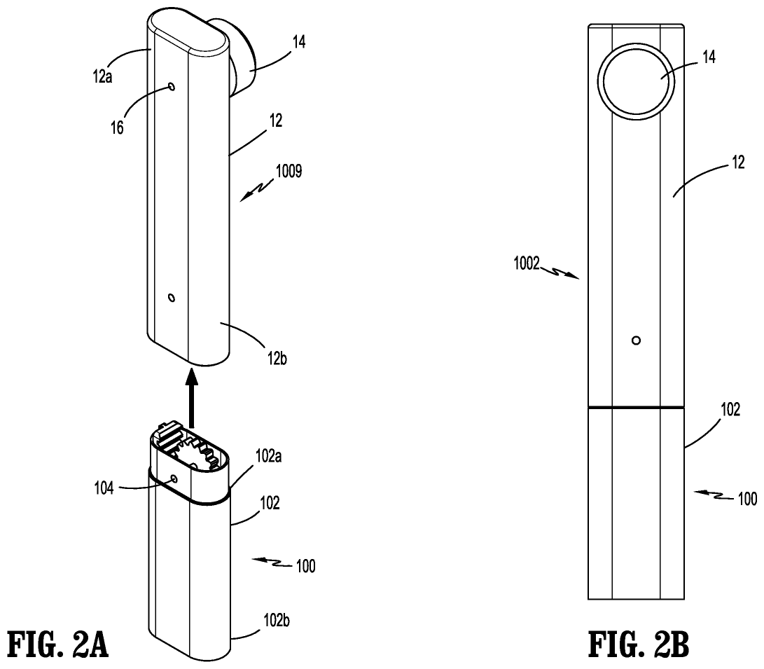

With reference to FIGS. 2A and 2B, the attaching device 1009 of the robotic arm 1002 includes an elongated housing 12 and the surgical instrument 100 includes an elongated housing 102 configured to detachably couple to the elongate housing 12 of the robotic arm 1002. The housing 12 of the robotic arm 1002 has a proximal end portion 12a supporting a motor 14, such as, for example, a servomotor, and a distal end portion 12b configured to detachably receive a proximal end portion 102a of the housing 102 of the surgical instrument 100. The motor 14 may be in communication with the control device 1004 (FIG. 1) such that a clinician may activate the motor 14 via the manual input devices 1007, 1008.

The distal end portion 12b of the housing 12 of the robotic arm 1002 may have a mechanical mating feature 16, such as, for example, a projection or an aperture, and the proximal end portion 102a of the housing 102 of the surgical instrument 100 may have a corresponding mechanical mating feature 104, which is the other of the projection or aperture. As such, upon inserting the proximal end portion 102a of the housing 102 of the surgical instrument 100 into the distal end portion 12b of the housing 12 of the robotic arm 1002, the projection 16 or 104 is received in the aperture 16 or 104 to fix the surgical instrument 100 to the robotic arm 1002. In other aspects, the housing 102 of the surgical instrument 100 may receive the distal end portion 12b of the housing 12 of the robotic arm 1002. Other mechanisms for detachably fastening the housings 12, 102 together are also contemplated, such as, for example, a magnetic coupling, fasteners, adhesive, friction-fit, latches, or the like.

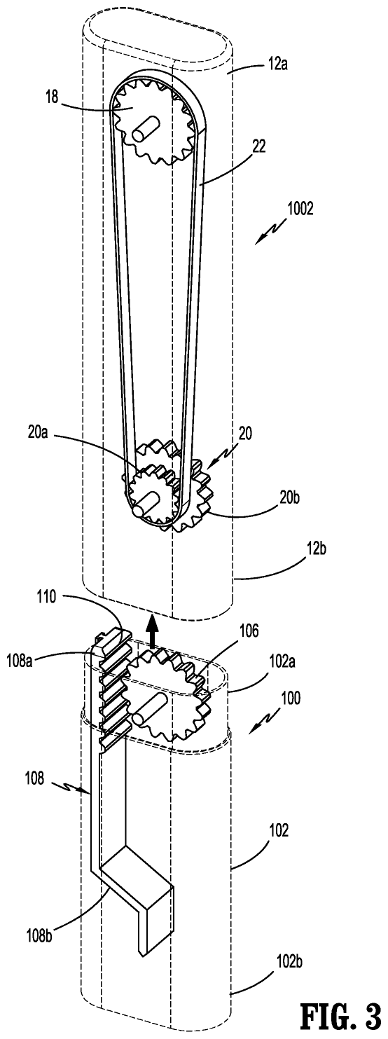

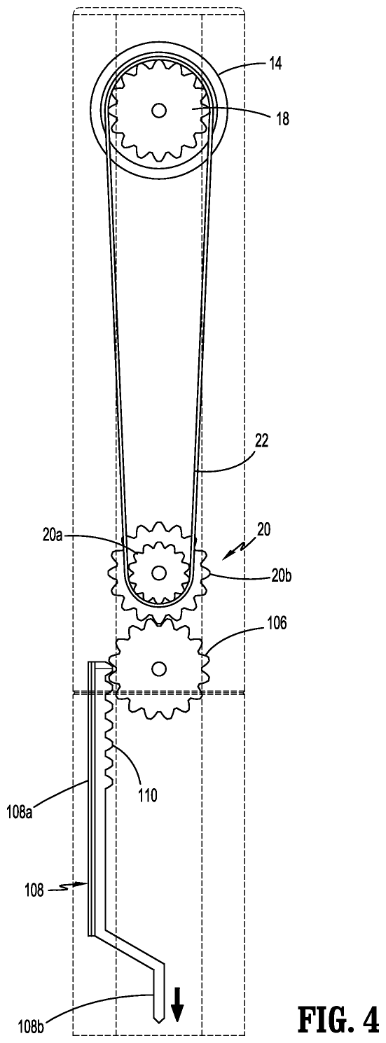

With reference to FIGS. 3 and 4, the robotic arm 1002 includes a first pulley 18, a second pulley 20, and a toothed belt 22 operably coupling the first and second pulleys 18, 20 to one another. The first pulley 18 is rotationally supported in the proximal end portion 12a of the housing 12 and is drivingly coupled to the motor 14 (FIGS. 2A and 2B). The first pulley 18 may be an annular gear, such as, for example, a spur gear. In aspects, the first pulley 18 may be alternatively configured as a friction pulley. The second pulley 20 is rotationally supported in the distal end portion 12b of the housing 12 and has a first rotating part 20a and a second rotating part 20b rotationally fixed to the first rotating part 20a. The second pulley 20 may be a compound gear such that the first rotating part 20a is a first gear (e.g., a spur gear) and the second rotating part 20b is a second, larger gear (e.g., a spur gear) rotationally fixed to the first gear 20a.

The first gear 20a is operably coupled to the first pulley 18 via the belt 22, which surrounds and meshingly engages gear teeth of the first pulley 18 and gear teeth of the first gear 20a of the second pulley 20. The belt 22 is configured to transfer rotational motion of the first pulley 18 to the second pulley 20. In aspects where the first pulley 18 and the first rotating part 20a of the second pulley 20 are each configured as friction pulleys, the belt 22 may be devoid of teeth and instead frictionally engage a smooth outer annular surface of the first pulley 18 and a smooth outer annular surface of the first rotating part 20a.

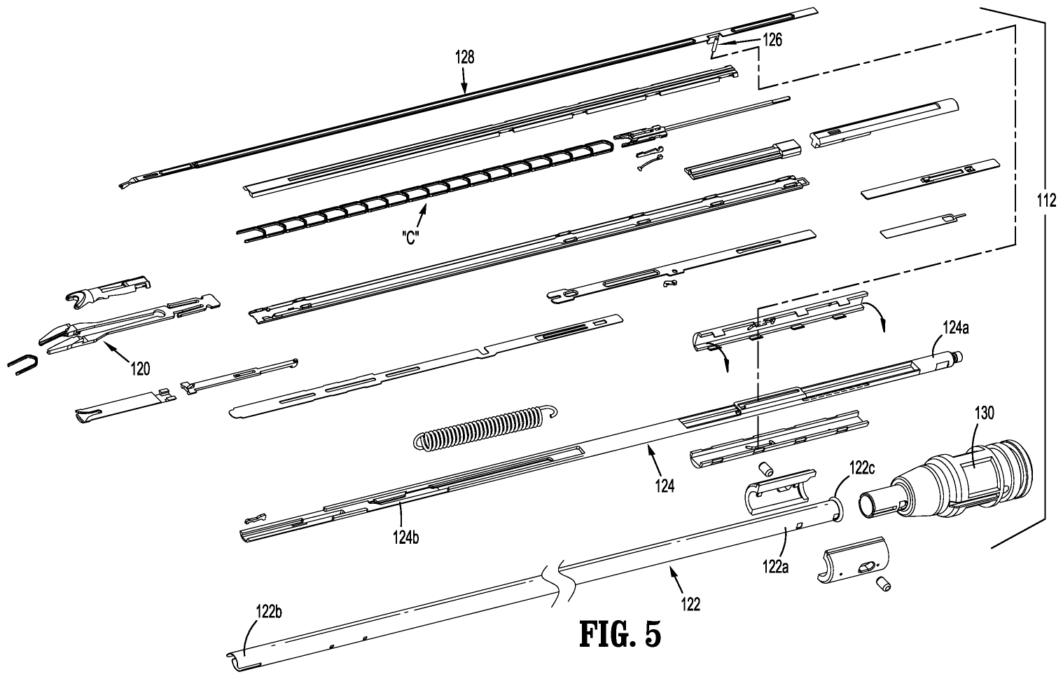

With continued reference to FIGS. 3 and 4, the surgical instrument 100 includes an annular gear 106, such as, for example, a pinion gear, rotationally supported in the proximal end portion 102a of the housing 102 thereof. The annular gear 106 is configured to operably engage the second gear 20b of the second pulley 20 of the surgical robotic arm 1002 upon coupling the housing 102 of the surgical instrument 100 to the housing 12 of the surgical robotic arm 1002. The annular gear 106 may protrude proximally from the proximal end portion 102a of the housing 102 to facilitate engagement between the annular gear 106 of the surgical instrument 100 and the second gear 20b of the robotic arm 1002. The annular gear 1006 is configured to transfer rotational motion from the first pulley 18 of the surgical robotic arm 1002 to a functional component of an end effector 112 (FIG. 5) of the surgical instrument 100. It is contemplated that the diameter of the first and second gears 20a, 20b and the annular gear 106 may be altered to optimize the total strength and stroke length needed to fire an implantable element (e.g., a surgical clip or staple).

The surgical instrument 100 further includes a linear actuator 108 supported in the housing 102 of the surgical instrument 100 and configured to move axially within the housing 102 in response to a rotation of the annular gear 106. The linear actuator 108 may have an elongated proximal end portion 108a in the form of a rack and a bent distal end portion 108b. The proximal end portion 108a of the linear actuator 108 is operably engaged with the annular gear 106. For example, the proximal end portion 108a of the linear actuator 108 may have a linear array of gear teeth 110 in meshing engagement with the annular gear 106 so that rotational motion of the annular gear 106 results in linear motion of the linear actuator 108. The distal end portion 108b of the linear actuator 108 is coupled to the functional component of the end effector 112 of the surgical instrument 100, such as, for example, a spindle 124 (FIG. 5) of a clip applier end effector 112, as will be described in further detail. In aspects, the surgical instrument 100 may have any suitable end effector suitable for performing a particular surgical procedure, such as a linear or circular stapler, a tack applier, or the like.

FIG. 5 illustrates an exemplary embodiment of an end effector 112 of the surgical instrument 100. The end effector 112 may be a clip applier that stores a stack of surgical clips “C” therein and has a pair of jaws 120 configured to form, in seriatim, the surgical clips “C” received from a pusher bar 128 of the end effector 112 upon receiving linear motion from the linear actuator 108 (FIG. 4) of the surgical instrument 100. The end effector 112 includes an elongated outer member or outer tube 122, an elongated spindle or inner shaft 124 axially movable within the outer tube 122 for effecting the stapling function of the end effector 112, and a slidable member 126 movably coupled to the spindle 124 for axially translating the pusher bar 128 to load and hold the surgical clips “C” in the jaws 120 during clip formation.

The outer tube 122 has a proximal portion 122a supported and secured to a hub 130, and a distal portion 122b supporting the jaws 120. The hub 130 may be configured to be coupled, either permanently or detachably, to the distal end portion 12b of the housing 102 (FIGS. 2A-4) of the surgical instrument 100. It is contemplated that the hub 130 may be sized and shaped for a friction-fit engagement with the distal end portion 12b of the housing 12. The outer tube 122 defines a lumen 122c extending longitudinally therethrough dimensioned for slidable receipt of the spindle 124.

The spindle or inner shaft 124 of the shaft assembly 100 is slidably supported within the lumen 122c of the outer tube 122 and has a generally elongated configuration. The spindle 124 includes a proximal portion 124a, and a distal portion 124b configured to selectively actuate the pair of jaws 120 during distal advancement of the spindle 124. The proximal portion 124a of the spindle 124 may define a hook, an enlarged head or other translational force coupling feature configured to be coupled to the distal end portion 108b of the linear actuator 108 of the surgical instrument 100. For example, the proximal portion 124a of the spindle 124 may be permanently or detachably fixed to the distal end portion 108 of the linear actuator 108 via a fastener, adhesive, a friction fit engagement, a latch, a bayonet-type connection, or the like.

For a more detailed description of certain aspects of the surgical clip applier end effector 112, reference may be made to U.S. patent application Ser. No. 16/042,227, filed on Jul. 23, 2018, the entire contents of which are incorporated by reference herein.

In operation, with initial reference to FIGS. 2A and 2B, the surgical instrument 100 is translated toward the robotic arm 1002 to insert the proximal end portion 102a of the housing 102 of the surgical instrument 100 into the housing 12 of the robotic arm 1002. The mating features 104, 16 of the housings 102, 12 of the respective surgical instrument 100 and robotic arm 1002 mate with one another and mechanically fix the surgical instrument 100 to the robotic arm 1002, as shown in FIG. 2B. With reference to FIGS. 3 and 4, upon engaging the housings 102, 12 of the respective surgical instrument 100 and robotic arm 1002, the annular gear 106 of the surgical instrument 100 operably engages the second gear 20b of the second pulley 20 of the robotic arm 1002.

With the annular gear 106 of the surgical instrument 100 operably engaged with the second pulley 20 of the robotic arm 1002, the drive motor 14 of the robotic arm 1002 and the linear actuator 108/spindle 124 of the surgical instrument 100 are operably coupled to one another. Accordingly, an activation of the drive motor 14 of the surgical robotic arm 1002 results in a translation of the spindle 124 (FIG. 5) of the end effector 112. In particular, an activation of the drive motor 14 rotates the first pulley 18 and, in turn, the belt 22, and the first and second gears 20a, 20b of the second pulley 20. Since the second gear 20b of the second pulley 20 is operably engaged with the annular gear 106 of the surgical instrument 100, the rotation of the second gear 20b causes a rotation of the annular gear 106. Due to the teeth 110 of the linear actuator 108 being engaged with the annular gear 106, the rotation of the annular gear 106 drives a translation or axial movement of the linear actuator 108 within and relative to the housing 102 in either a proximal or distal direction depending on the intent of the user or control device 1004 (FIG. 1). Since the proximal end portion 124a (FIG. 5) of the spindle 124 of the end effector 112 is axially restrained to the distal end portion 108b of the linear actuator 108, the spindle 124 is driven either proximally or distally by the translational movement of the linear actuator 108. In aspects, a distal translation of the spindle 124 may effect a stapling function of the end effector 112 and a subsequent proximal translation of the spindle 124 may effect a reset of the end effector 112.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical robotic system for use in a minimally invasive surgical procedure, comprising:
a surgical robotic arm including:
a housing having a proximal end portion and a distal end portion;
a first pulley rotationally supported in the proximal end portion of the housing;
a second pulley rotationally supported in the distal end portion of the housing; and
a belt operably coupling the first and second pulleys to one another; and
a surgical instrument including:
a housing having a proximal end portion configured to couple to the distal end portion of the housing of the surgical robotic arm; and
a gear rotationally supported in the proximal end portion of the housing of the surgical instrument and configured to operably couple to the second pulley of the surgical robotic arm, wherein the gear is configured to transfer rotational motion from the first pulley of the surgical robotic arm to a functional component of an end effector of the surgical instrument.

2. The surgical robotic system according to claim 1, wherein the second pulley is a compound gear including:
a first gear operably engaged to the belt and configured to rotate in response to movement of the belt; and
a second gear non-rotatably fixed to the first gear and configured to operably engage the gear of the surgical instrument.

3. The surgical robotic system according to claim 2, wherein at least one of the gear of the surgical instrument protrudes proximally from the proximal end portion of the housing of the surgical instrument or the second gear of the surgical robotic arm protrudes distally from the distal end portion of the housing of the surgical robotic arm.

4. The surgical robotic system according to claim 1, wherein the surgical instrument further includes a linear actuator supported in the housing of the surgical instrument and configured to move axially within the housing in response to a rotation of the gear.

5. The surgical robotic system according to claim 4, wherein the linear actuator has a proximal end portion operably engaged with the gear, and a distal end portion configured to couple to the functional component of the end effector.

6. The surgical robotic system according to claim 5, wherein the proximal end portion of the linear actuator has a plurality of gear teeth in meshing engagement with the gear.

7. The surgical robotic system according to claim 1, wherein the surgical robotic arm further includes a motor drivingly coupled to the first pulley and configured to rotate the first pulley.

8. The surgical robotic system according to claim 1, wherein the distal end portion of the housing of the surgical robotic arm has a male or female mechanical mating feature, and the proximal end portion of the housing of the surgical instrument has the other of the male or female mechanical mating feature configured to matingly engage the male or female mechanical mating feature of the distal end portion of the housing of the surgical robotic arm.

9. A surgical robotic system for use in a minimally invasive surgical procedure, comprising:
a surgical robotic arm including:
an elongated housing having a proximal end portion and a distal end portion;
a pulley rotationally supported in the proximal end portion of the housing; and
a compound gear rotationally supported in the distal end portion of the housing and operably engaged to the pulley; and
a surgical instrument including:
an elongated housing having a proximal end portion configured to detachably couple to the distal end portion of the housing of the surgical robotic arm;
an annular gear rotationally supported in the proximal end portion of the housing of the surgical instrument and configured to operably couple to the compound gear of the surgical robotic arm; and
a linear actuator supported in the housing of the surgical instrument and having a proximal end portion operably engaged to the annular gear, and a distal end portion configured to couple to a functional component of an end effector of the surgical instrument, wherein the linear actuator is configured to move axially within the housing of the surgical instrument in response to a rotation of the pulley of the surgical robotic arm when the surgical instrument is coupled to the surgical robotic arm.

10. The surgical robotic system according to claim 9, wherein the compound gear includes:
a first gear operably coupled to the pulley; and
a second gear non-rotatably fixed to the first gear and configured to operably engage the annular gear of the surgical instrument.

11. The surgical robotic system according to claim 10, wherein the surgical robotic arm further includes a belt wrapped around and engaged to the pulley and the first gear, the belt being configured to transfer rotational motion of the pulley to the compound gear.

12. The surgical robotic system according to claim 11, wherein at least one of the annular gear protrudes proximally from the proximal end portion of the housing of the surgical instrument or the second gear protrudes distally from the distal end portion of the housing of the surgical robotic arm.

13. The surgical robotic system according to claim 9, wherein the proximal end portion of the linear actuator has a plurality of gear teeth in meshing engagement with the annular gear.

14. The surgical robotic system according to claim 9, wherein the surgical robotic arm further includes a motor drivingly coupled to the pulley and configured to rotate the pulley.
